(19) United States
(12) Patent Application Publication
(10) Pub. No.: US 2023/0248675 A1
(43) Pub. Date: Aug. 10, 2023

(54) SOLID DISPERSION FOR THERAPEUTIC USE

(71) Applicant: Florida Research Group, LLC, Tampa, FL (US)

(72) Inventor: Kotha SEKHARAM, Tampa, FL (US)

(21) Appl. No.: 18/159,806

(22) Filed: Jan. 26, 2023

Related U.S. Application Data
(60) Provisional application No. 63/303,250, filed on Jan. 26, 2022.

(51) Int. Cl. A61K 31/19; A61K 9/16

(52) U.S. Cl. CPC A61K 31/19; A61K 9/1694; A61K 9/1635

(57) ABSTRACT

The present invention relates to a solid dispersion with improved bioavailability of an insoluble medicament, particularly a solid dispersion of the insoluble medicament, 3β-hydroxyurs-12-en-28-oic acid, and a preparation method thereof.

### CROSS REFERENCE TO A RELATED APPLICATION

This application claims the benefit of U.S. Provisional Application Ser. No. 63/303,250, filed Jan. 26, 2022, which is incorporated herein by reference in its entirety.

### BACKGROUND OF THE INVENTION

3β-hydroxyurs-12-en-28-oic acid, also called ursolic acid is a naturally-derived lipophilic pentacyclic triterpene, found mostly in rosemary, marjoram, lavender, thyme, and organum, apple fruit peel and has a chemical formula of C₃₀H₄₈O₃, and a melting point of 283-285° C.

3β-hydroxyurs-12-en-28-oic acid has a wide range of therapeutic benefits as a cytokinin-modulator' to help mitigate effects of COVID virus infection, and also as an anti-oxidant, anti-inflammatory, anti-cancer, anti-allergic, hepatoprotective, gastroprotective, hypolipidemic, hypoglycemic, lipolytic anti-obesity, anti-atherogenic and immunomodulatory effects.

3β-hydroxyurs-12-en-28-oic acid is highly water-insoluble. Limited solubility, poor bioavailability and rapid metabolism of 3β-hydroxyurs-12-en-28-oic acid have limited its clinical applications for therapeutic use.

Bioavailability can be improved by several methods, including chemical modification (prodrug, salt formation), physical modification (nano crystals, co-crystals, loading on porous materials, solid dispersions), carrier system (cyclodextrins, emulsions, microemulsions, liposomes).

A reduction in particle size improves the dissolution rate significantly. Wet milling and nano-technology are two techniques that can be applied to improve absorption to a certain extent. However, difficulty in scalability and contaminants from milling equipment limit commercial usage.

Solubilization can be improved by particle size reduction, pH adjustment, salt formation, solid dispersion, complexation, co-solvency, micellization, or a combination effect of any of the above. Processing methods involving usage of solvents is highly unfavorable due to residual solvents, environmental concerns and challenges in scalability limit their application in production process.

Hot melt extrusion (HME) is a process wherein an active ingredient and polymeric materials are blended and passed through an extruder with high pressure and defined temperature zones, particularly above their glass transition temperature (Tg) for molecular level mixing of thermoplastic binders and/or polymers and active compounds. The goal is to maintain the active ingredient in this amorphous state and prevent any phase reversal. There is no need for any solvents and the process is simple with fewer processing steps.

Free energy of non-crystalline solids is much higher than that of their corresponding crystalline forms. As a result of its higher internal energy, during processing or storage the amorphous state may spontaneously convert back to the crystalline state, as reported by Danni Yu et al. (2020).

Danni Yu et al.; reported that crystalline 3β-hydroxyurs-12-en-28-oic acid exhibited poor physical stability and it underwent structural change after 30 days of storage under 25° C./75% RH or 40° C. /75% RH.

Danni Yu et al; also reported that amorphous 3β-hydroxyurs-12-en-28-oic acid prepared by ball milling exhibited a nearly complete conversion toward its stable crystalline form after 8 days of storage at both 25° C. 75% RH or 40° C. /75% RH [Triple Strategies to Improve Oral Bioavailability by Fabricating Co-amorphous Forms of Ursolic Acid with Piperine: Enhancing Water-Solubility, Permeability and Inhibiting Cytochrome P450 Isozymes. Danni Yu et al; Mol. Pharmaceutics, Publication Date (Web): 14 Sep. 2020].

In spite of several advantages, the HME process has its own disadvantages. Three main obstacles limiting the commercial application of HME are (1) thermal degradation of heat-sensitive active ingredients at high process temperature, (2) recrystallization of amorphous drugs during storage and dissolving process, and (3) difficulty to obtain products with reproducible physicochemical properties.

### BRIEF SUMMARY OF THE INVENTION

The present invention provides a solid dispersion that improves the solubility and bioavailability of insoluble medicament 3β-hydroxyurs-12-en-28-oic acid and a preparation method and uses thereof.

### BRIEF DESCRIPTION OF THE DRAWINGS

FIG. 1 shows an X-ray diffractometry of [A] unprocessed 3β-hydroxyurs-12-en-28-oic acid indicating crystalline nature [B] processed 3β-hydroxyurs-12-en-28-oic acid indicating amorphous nature [C] processed −2 sample of 3β-hydroxyurs-12-en-28-oic acid indicating amorphous nature.

FIG. 2 shows mean plasma concentration profile of test compound following oral administration of oral dose in Beagle dogs at a dose of 50 mg/dog. G1 Untreated test compound. G2 Treated compound by method-A. G3: Treated compound by method-B.

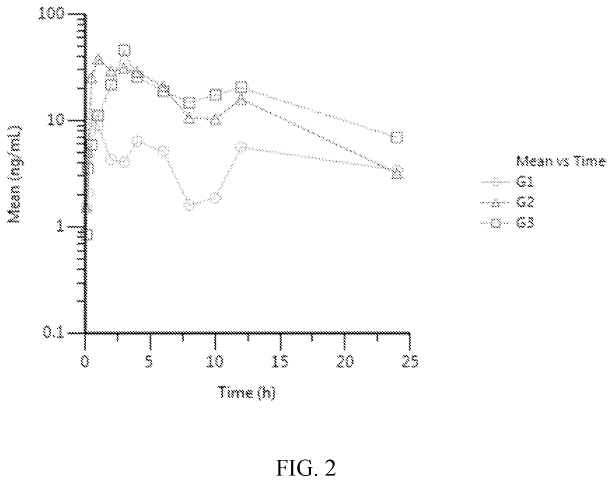

FIG. 3 shows irreversible stability of 3β-hydroxyurs-12-en-28-oic acid processed according to the process described in this invention. Samples A, B & C are stored between 3 to 15 months. All 3 samples are in amorphous state and show no reversal to crystalline state, proving the merit of this invention.

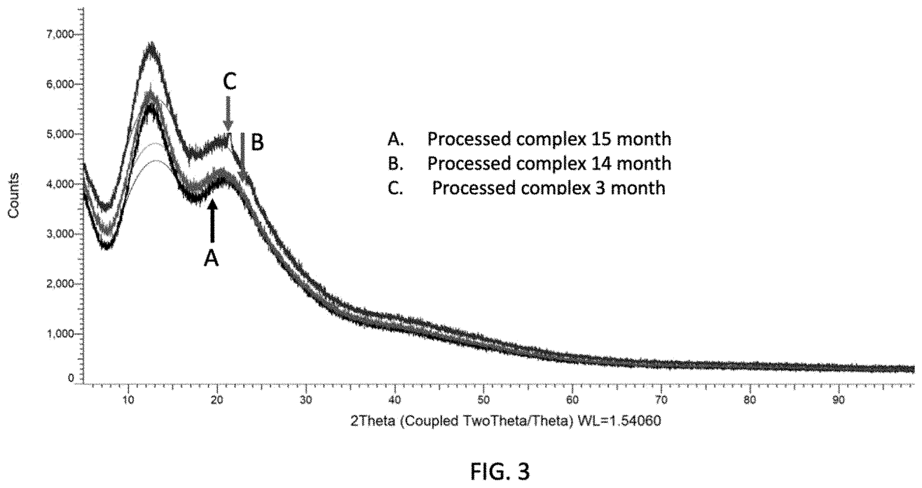

### DETAILED DESCRIPTION OF THE INVENTION

The present invention provides a solid dispersion comprising 3β-hydroxyurs-12-en-28-oic acid as an active ingredient and a water-soluble polymer having glass transition temperature lower than 284° C. and is prepared by melt extrusion.

The present invention further provides a method for preparing the solid dispersion by blending active ingredient 3β-hydroxyurs-12-en-28-oic acid with a water soluble polymer having glass transition temperature lower than 284° C. and is prepared by melt extrusion.

In certain embodiments, the melt extrusion is performed at a temperature from about 50° C. to about 200° C.

The active ingredient 3β-hydroxyurs-12-en-28-oic acid used in the present invention is a naturally-derived lipophilic pentacyclic triterpene acid with chemical formula is C₃₀H₄₈O₃, and a melting point of 283-285° C. In the present invention, the term 3β-hydroxyurs-12-en-28-oic acid is used as comprising all the pharmaceutically acceptable salts, isomers, solvates, extracts containing 3β-hydroxyurs-12-en-28-oic acid and a combination thereof.

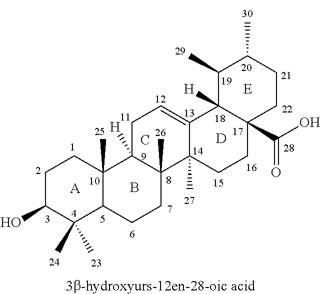

The active ingredient 3β-hydroxyurs-12-en-28-oic acid may be used to cure and prevent a medical condition. With anti-inflammatory, anti-oxidant, anti-apoptotic, and anti-carcinogenic effects, 3β-hydroxyurs-12-en-28-oic acid may be useful to prevent or treat certain types of cancer, obesity/diabetes, cardiovascular disease, brain disease, liver disease, and muscle wasting, if bioavailability is at acceptable level.

In some embodiments, the bioavailability of 3β-hydroxyurs-12-en-28-oic acid in a final composition prepared according to the present invention has improved at least two folds over unprocessed 3β-hydroxyurs-12-en-28-oic acid. In further embodiments, the improvement is at least four folds.

The active ingredient 3β-hydroxyurs-12-en-28-oic acidic is contained preferably in the amount of about 10 to about 70 wt %, and more preferably about 5% to about 50% based on the total weight of the final composition. Because of low density, at 10% level, it is not difficult to achieve content uniformity in the blend.

In the solid dispersion of the present invention, the carrier may include a water-soluble polymer having a glass transition temperature below 284° C., which preferably is Polyvinyl caprolactampolyvinyl acetate-polyethylene glycol graft copolymer (Soluplus®), polyvinylpyrrolidone-vinyl acetate copolymer (PVP VA64), polyethylene glycol (PEG), Eudragit® EPO, and hypromellose acetate succinate (HPMCAS). In some embodiments, a water-soluble polymer is about 5% to about 50% of the weight of the final composition. The water-soluble polymer is contained preferably in the amount of 30 wt % or more, more preferably in the amount of 30 to 90 wt %, based on the total weight of the final composition. Polyvinyl pyrrolidone having any molecular weight can be used, but in particular those having the molecular weight of 45,000 to 70,000 are desired because they may build the viscosity highly suitable for melt-extrusion. More preferable polymer is vinylpyrrolidone-vinyl acetate copolymer. Other polymers can also be used.

In one embodiment, the present invention does not need a plasticizer. However, usage of a plasticizer including D-alpha-tocopheryl polyethylene glycol 1000 succinate (TPGS), polyethylene glycol 400 and other customary plasticizers are not restricted.

As preferable embodiments of the present invention, there are provided solid dispersions comprising the active ingredient 3β-hydroxyurs-12-en-28-oic acidic in 10 to 50 wt % and the water-soluble polymer polyvinyl pyrrolidone in 50 to 90 wt %, or comprising the active ingredient 3β-hydroxyurs-12-en-28-oic acidic in 10 to 50 wt %, the water-soluble polymer polyvinyl pyrrolidone or hypromellose acetate succinate in 45 to 85 wt %.

The blend prepared as above is melted when it passes through four (4) or more heating zones whose temperature is sequentially lowered. Specifically, the mixture of active ingredient and water-soluble polymer that have been mixed in advance as a powder is introduced into an extruder and melt-extruded to prepare the solid dispersion of the present invention wherein the extruder is made of several heating zones designed to be distinguished from each other and which are connected in series. Here, the distinguished heating zones are controlled to have a temperature lower than the melting point (284° C.) of the melted 3β-hydroxyurs-12-en-28-oic acid. In some embodiments, this melt extrusion of the mixture is performed at a temperature ranging from about 50° C. to about 200° C. More preferably, the heating zones consist of the first to fourth heating zones wherein the temperature of the first heating zone is controlled to 160 to 145° C., that of the second heating zone to 144 to 120° C., that of the third heating zone to 119 to 80° C., and that of the fourth heating zone to 79 to 70° C.

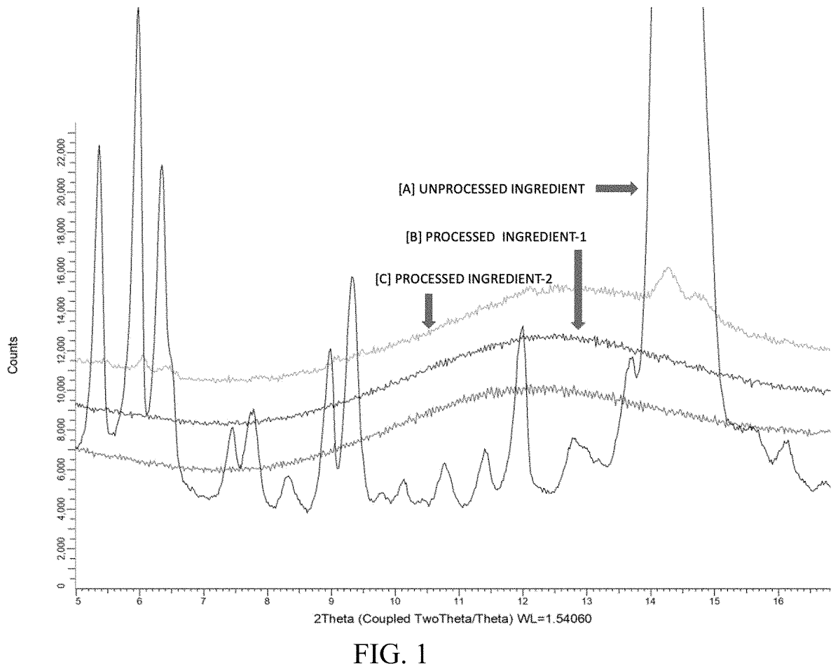

Although the melting is performed at a temperature lower than the melting point, the extrusion is performed through the specific sustained cooling and sequential melting by passing through the several heating zones whose temperatures are lowered sequentially causing molecular changes to the active ingredient, causing conversion of crystalline structure to amorphous form. As illustrated in the FIG. 1, the active ingredient processed according to the method described in this invention clearly shows its conversion to amorphous form.

The melt extruded product is ground to fine powder by using a mill, further processed and/or blended with excipients to form a pharmaceutical composition such as a tablet, capsule, chew, gummy, lozenge, powder for sprinkle formulations, or suppository.

Customary excipients include, but are not limited to, flow agents to fill in to oral capsules, tableting agents, disintegrants to form tablets, flavoring agents, sweeteners to form chews and solidified fats to form suppositories.

Pharmaceutical compositions as used herein are meant for administration to mammals including primates. Preferably pharmaceutical compositions according to the present invention are administered to humans.

### EXAMPLE 1—MFTHOD OF MANUFACTURING

An extract containing 3β-hydroxyurs-12-en-28-oic acid is mixed with vinylpyrrolidone-vinyl acetate copolymer at a ratio of 10:90 to form a uniform physical mixture (PM). This PM was passed through hot melt extruder (Thermofisher, Steer) at temperature 160° C. The extrudate is collected from the die and cooled at ambient temperature and pulverized to 80 mesh.

The extrudate material was tested to confirm the conversion of crystalline structure of the compound to amorphous form and it maintained the status on storage.

This processed extract is formulated to a pharmaceutical composition such as a capsule, tablet, chewable tablet, soft chew, gummy, lozenge, powder for sprinkle formulations, or suppository, delivering desired amount of active ingredient made with solid dispersion made according to the described method.

### EXAMPLE 2—PHARMACOKINETIC STUDIES

The objective of the study was to investigate pharmacokinetics of test compound in dogs following oral administration of a solid dose of test compound (50 mg).

A total of 9 Beagle dogs (3 animal/group) were allotted for the study. One dose containing 50 mg active was mixed in dog feed and provided to respective dogs. Dosing time was noted after consuming total feed.

Post dose, approximately 1.0 mL of blood sample/animal/time point were collected from each dog via cephalic vein into pre-labeled Eppendorf tubes containing anticoagulant 10% K2 EDTA. Blood was collected at the following time points viz; at pre-dose, 5 min, 15 min, 30 min, 1 h, 2 h, 3 h, 4 h, 6 h, 8 h, 10 h, 12 h and 24 h after dosing. Collected blood samples were mixed gently and kept on cool packs. Blood samples were centrifuged at 3,500 rpm for 10 min at 4° C. Plasma separation was carried out within 1 hour of blood sample collection and stored at −70±10° C. until analysis. PK parameters were evaluated using Phoenix WinNonlin Ent-version 8.2 by non-compartmental analysis.

Data indicate that two treated test solid dispersion products, when compared to untreated product have four-fold increase in bioavailability. This confirms utility of the solid dispersion composition made according to the method disclosed here. In addition, the active ingredient processed through present method was found to be (1) thermally stable (2) retained amorphous form without any recrystallization and (3) obtained consistent results in repeated experiments. This validates the value of present process of preparing the dispersion and the therapeutic dose made with the dispersion.

In the solid dispersion of the present invention, the carrier may include a water-soluble polymer having a glass transition temperature below 284° C., which preferably is Polyvinyl caprolactampolyvinyl acetate-polyethylene glycol graft copolymer (Soluplus®), polyvinylpyrrolidone-vinyl acetate copolymer

(PVP VA64), polyethylene glycol (PEG), Eudragit® EPO, and hypromellose acetate succinate (HPMCAS), The water-soluble polymer is contained preferably in the amount of 30 wt % or more, more preferably in the amount of 30 to 90 wt %, based on the total weight of the composition. Polyvinyl pyrrolidone having any molecular weight can be used, but in particular those having the molecular weight of 45,000 to 70,000 are desired since they may build the viscosity highly suitable for melt-extrusion. More preferable polymer is vinylpyrrolidone-vinyl acetate copolymer. Other polymers can also be used.

The present invention may not need a plasticizer. However, usage of a plasticizer including D-alpha-tocopheryl polyethylene glycol 1000 succinate (TPGS), polyethylene glycol 400 and other customary plasticizers are not restricted.

As preferable embodiments of the present invention, there are provided solid dispersions comprising the active ingredient 3β-hydroxyurs-12-en-28-oic acidic in 10 to 50 wt % and the water-soluble polymer polyvinyl pyrrolidone in 50 to 90 wt %, or comprising the active ingredient 3β-hydroxyurs-12-en-28-oic acidic in 10 to 50 wt %, the water-soluble polymer polyvinyl pyrrolidone or hypromellose acetate succinate in 45 to 85 wt %.

The blend prepared as above is melted when it passes through four (4) or more heating zones whose temperature is sequentially lowered. Specifically, the mixture of active ingredient and water-soluble polymer which have been mixed in advance as a powder is introduced into an extruder and melt-extruded to prepare the solid dispersion of the present invention wherein the extruder is made of several heating zones designed to be distinguished from each other and which are connected in series. Here, the distinguished heating zones are controlled to have a temperature lower than the melting point (284° C.) of the melted 3β-hydroxyurs-12-en-28-oic acid. More preferably, the heating zones consist of the first to fourth heating zones wherein the melting temperature of the first heating zone is controlled to 160 to 145° C., that of the second heating zone to 144 to 120° C., that of the third heating zone to 119 to 80° C., and that of the fourth heating zone to 79 to 70° C.

Although the melting is performed at a temperature lower than the melting point, the extrusion is performed through the specific sustained cooling and sequential melting by passing through the several heating zones whose setting temperatures are lowered sequentially causing molecular changes to the active ingredient, causing conversion of crystalline structure to amorphous form. As illustrated in the FIG. 1, the active ingredient processed according to the method described in this invention clearly shows its conversion to amorphous form.

The melt extruded product is ground to fine powder by using a mill, further processed and/or blended with excipients to form a pharmaceutical composition such as a tablet, capsule, chew, gummy, lozenge, powder for sprinkle formulations, or suppository.

Customary excipients include, but not limited to, flow agents to fill in to oral capsules, tableting agents, disintegrants to form tablets, flavoring agents, sweeteners to form chews and solidified fats to form suppositories.

TABLE 1
HME Parameters

| Parameters | |
|---|---|
| Extruder | Thermofisher Pharmalb 16 |
| | Steer Twin screw 20 |
| Zone 5 temperature | 27° C. |
| Zone 4 temperature | 40° C. |
| Zone 3 temperature | 80° C. |
| Zone 2 temperature | 110° C. |
| Zone 1 temperature | 150° C. |
| Screw speed (RPM) | 300 |
| Torque (%) | 50-57 |
| Feed rate (g/min) | 6 |

TABLE 2
Mean pharmacokinetic parameters of test compound, treatment1 and treatment-2

| | Control (with | | | | | |
|---|---|---|---|---|---|---|
| PK | untreated extract) | | Treatment-1 | | Treatment-2 | |
| parameters | Mean | SD | Mean | SD | Mean | SD |
| Tₘₐₓ (h) @ | 3 | NR | 2 | NR | 3 | NR |
| Cₘₐₓ (ng/mL) | 12.51 | 14.03 | 58.85 | 27.41 | 53.91 | 32.59 |
| AUC₀₋₂₄ h | 101.12 | 131.36 | 358.04 | 157.05 | 398.92 | 233.99 |
| (ng · h/mL) | | | | | | |
| AUC_{0-inf} | 2242.65# | NA | 390.96 | 158.83 | 374.76$ | NA |
| (ng · h/mL) | | | | | | |
| T_{1/2} (h) | 134.65# | NA | 6.62 | 1.97 | 3.21 | NA |
| MRT last | 7.46 | 4.19 | 7.61 | 1.32 | 8.41 | 4.77 |

1. A process for preparing a solid dispersion for improved therapeutic use, comprising:
i) blending 3β-hydroxyurs-12-en-28-oic acid, and a water-soluble polymer having a glass transition temperature lower than 284° C.;
ii) melt extruding the mixture at a temperature from about 50° C. to about 200° C.;
iii) grinding the melt extrudate; and
iv) blending the milled extrudate with excipients to form a pharmaceutical composition;
v) wherein said process is free from any solvent-associated processing steps; and
vi) said pharmaceutical composition has an increased bioavailability of at least two-folds over unprocessed material.

2. The process of claim 1, wherein the 3β-hydroxyurs-12-en-28-oic acid is a compound of formula:

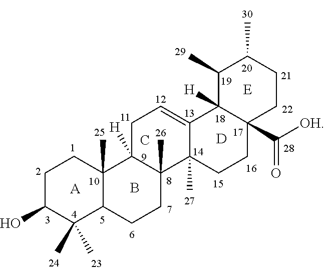

3. The process of claim 1, wherein the 3β-hydroxyurs-12-en-28-oic acid is a chemical isolate, or salt or an extract containing 3β-hydroxyurs-12-en-28-oic acid.

4. The process of claim 1, wherein the water soluble polymer is selected from the group consisting of polyvinyl caprolactampolyvinyl acetate-polyethylene glycol graft copolymer, polyvinylpyrrolidone-vinyl acetate copolymer, polyethylene glycol, Eudragit® EPO, hypromellose acetate succinate and combination thereof.

5. The process of claim 1, wherein the water-soluble polymer is poly vinylpyrrolidone-vinyl acetate copolymer.

6. The process of claim 1, wherein said 3β-hydroxyurs-12-en-28-oic acid is about 5% to about 50% of the weight of the pharmaceutical composition as a finished product.

7. The process of claim 1, wherein said water-soluble polymer is about 5% to about 50% of the weight of the pharmaceutical composition as a finished product.

8. The process of claim 1, wherein the blend of said 3β-hydroxyurs-12-en-28-oic acid and said water-soluble polymer is melted while it passes through at least four heating zones, wherein a temperature of the heating zone is sequentially lowered.

9. The process of claim 1, wherein the pharmaceutical composition prepared according to the method has bioavailability improved at least four folds over unprocessed material.

10. The process of claim 1, wherein the pharmaceutical composition is meant for mammals.
